(19) United States
(12) Patent Application Publication
(10) Pub. No.: US 2015/0033834 A1
(43) Pub. Date: Feb. 5, 2015

(54) APPARATUS FOR TESTING THE QUALITY OF A FLUID SAMPLE

(71) Applicants: Stephen Gundry, Bath (GB); Robert Edward Shenton Bain, Bristol (GB); Philip Walsh, Bristol (GB); Craig Ian Wightman, Bristol (GB); Robert Matthews, Bristol (GB); Thomas Martin Hilder, Bristol (GB)

(72) Inventors: Stephen Gundry, Bath (GB); Robert Edward Shenton Bain, Bristol (GB); Philip Walsh, Bristol (GB); Craig Ian Wightman, Bristol (GB); Robert Matthews, Bristol (GB); Thomas Martin Hilder, Bristol (GB)

(73) Assignee: The University of Bristol, Bristol (GB)

(21) Appl. No.: 14/002,582

(22) PCT Filed: Feb. 29, 2012
(86) PCT No.: PCT/GB2012/050452

§ 371 Date: Nov. 22, 2013

(30) Foreign Application Priority Data
Mar. 1, 2011 (GB) 1103482.4

(51) Int. Cl. G01N 33/18; G01N 1/10

(52) U.S. Cl. CPC G01N 33/18; G01N 1/10
USPC 73/64.56

(57) ABSTRACT

An apparatus for testing the quality of a fluid sample includes a chamber having an opening for receiving at least some of the fluid sample and a sealing element arranged to be movable to a sealing position. An insertion portion of the sealing element is received within an insertion space of the chamber to seal the opening One of the sealing element and container is arranged to define a displacement passage for enabling a portion of the fluid sample that is located within the insertion space to be displaced therefrom as the sealing element assumes the sealing position.

### BACKGROUND

As the Millennium Development Goals for water recognise, microbially contaminated drinking water is a major cause of diarrhoeal disease, responsible for the deaths of 1.8 million people every year (WHO, 2004) most of which are children in developing countries. In contrast, the development of new water testing technologies is driven by the needs of water companies in North America and Europe to adhere to the stringent standards set by regulatory authorities and, more recently, to concerns about bio-terrorism. Even with basic water testing equipment, skilled technicians and appropriate laboratory settings are rarely available in developing countries. As a result, there is a mismatch between the targets for technological development and the disease burden. This failure to develop appropriate diagnostics is analogous to the lack of investment by pharmaceutical companies to develop drugs to tackle diseases common only in developing countries.

When natural disasters occur, such as tsunami and earthquakes, agencies report that many of the attributable deaths are not the direct result of the disaster itself, but can be caused by subsequent outbreaks of disease, particularly from contaminated drinking water. Testing of drinking water sources after disasters presents particular problems due to the critical lack of staff, resources, and communications and transport infrastructure.

The World Health Organization issues Guidelines for Drinking-Water Quality. For bacteriological quality of drinking water, the WHO's Guidelines for drinking-water quality, state ‘(In) All water intended for drinking, E. coli or thermo tolerant coliform bacteria must not be detectable in any 100-ml sample’. Whilst adherence to this stringent standard is required and achieved by most developed countries, it is likely to be an unachievable target for most developing countries within the foreseeable future. This is particularly true where water is drawn from community sources in rural areas such as rivers or natural springs.

At present, many of the other available water testing technologies have been designed for use in developed countries. This is because the size of markets for water testing products is much greater in developed countries than in developing countries, where governments have only limited funds available for water testing. Many water testing technologies, such as the standard membrane filtration approach, require water samples to be collected in the field, stored under ice in transport containers, and then transported back to a microbiological laboratory. This microbiological laboratory needs to have appropriate facilities for testing samples, such as glassware incubators, lab benches, and facilities for the disposal of potentially hazardous waste, refrigerators, and trained technicians capable of undertaking water tests.

In remote areas of developing countries many of these facilities are simply unavailable. Ice for transporting water samples back to the laboratory may be impossible to obtain. The nearest microbiological laboratory may be a considerable distance away and there may be only very limited transport available for hard-pressed government environmental health technicians. Establishing a laboratory locally may also be difficult. Mains electricity may be either unavailable or available only sporadically and even buildings with workbenches and running water may be difficult to find. Many developing country organisations may be unable to afford the high consumables costs associated with some water tests. In many rural districts of developing countries, there is a lack of trained personnel able to carry out some of the more complex water testing procedures, such as calculating Most Probable Numbers of indicator bacteria or performing appropriate sample dilutions.

In recent years there has been some progress in the development of field kits for testing water. However, the present inventors have identified that known devices for testing water can suffer from one or more of the following disadvantages:

1. It can be difficult to partition a collected water sample into a plurality of individual subsamples, in particular when sealing is effected in a submerged condition.

2. It can be difficult to partition a collected water sample into a plurality of individual subsamples that remain isolated from one another throughout testing so as to avoid cross contamination between subsamples.

3. When testing for microbiological pathogens, it is desirable to keep the water sample isolated from the outside environment. However, devices that include moving parts to provide different configurations can encounter sealing problems that may result in a user being exposed to the microbiological pathogens and other organisms, which may be present in large numbers in the water sample.

4. Water testing devices that are arranged to isolate a water sample from the outside environment during testing can be prohibitive in terms of substances that can be detected and/or the reagents that can be used for the detection of substances.

There is therefore a need for apparatus for testing the quality of a fluid sample that substantially alleviates one or more of the above mentioned disadvantages.

### SUMMARY

In accordance with a first aspect of the present invention, there is provided an apparatus for testing the quality of a fluid sample, the apparatus including:
a chamber having a chamber wall having an opening for receiving at least some of the fluid sample; and
a sealing element arranged to be movable to a sealing position in which an insertion portion of the sealing element is received within an insertion space of the chamber to seal the opening,
wherein one of the sealing element and chamber wall is arranged to define a displacement passage for enabling a portion of the fluid sample that is located within the insertion space to be displaced therefrom as the sealing element assumes the sealing position.

Thus, the apparatus according to embodiments of the present invention is arranged to define a passageway that enables fluid occupying the space into which the sealing element will move to be displaced therefrom as the sealing element is moved to seal the chamber.

The sealing element may include a body portion having a peripheral sealing face which forms at least some of the insertion portion of the sealing element.

At least the insertion portion of the sealing element may be formed of a resiliently deformable material. The entire sealing element may be formed of a resiliently deformable material.

The displacement passage may be permanently formed in the sealing element and may include a second opening leading to an auxiliary chamber arranged to enable the portion of the fluid sample located within the insertion space to be displaced into the auxiliary chamber as the sealing element assumes the sealing position.

The second opening may be arranged to permit fluid of the fluid sample to enter the auxiliary chamber when the fluid is at a first pressure corresponding to the fluid pressure within the chamber as the sealing element assumes the sealing position, and may be arranged to inhibit fluid of the fluid sample from entering the auxiliary chamber when the fluid is at a second pressure less than the first pressure and corresponding to the fluid pressure within the chamber when the sealing element is spaced from the container.

The sealing element may include a head region that defines a portion of the auxiliary chamber, the head region being arranged so as not to enter the chamber when the sealing element is in the sealing position.

At least some of the head region may be formed of a resiliently deformable material such that the volume of the auxiliary chamber increases with an increase of fluid pressure within the chamber while the chamber is sealed by the sealing element.

The sealing element may be arranged such that net pressure resulting from an increase in fluid pressure within the sealed chamber acts against the container walls that define the insertion subspace of the chamber.

The displacement passage may be defined by a resiliently deformable displacement region of the sealing element or chamber wall, the resiliently deformable displacement region being arranged to deform as the sealing element assumes the sealing position to define a passageway for enabling the displacement of the portion of the fluid sample within the insertion space.

At least some of the chamber wall may be formed of a resiliently deformable material to form the resiliently deformable displacement region.

The apparatus may include a container defining an internal space for receiving the fluid sample, the chamber and sealing element being disposed within the internal space such that the chamber is filled with the fluid sample as the sealing element moves to the sealing position. The apparatus may include a plurality of chambers and a plurality of sealing elements, each of which may be disposed within the internal space. The container may include one or more transparent or translucent portions for enabling the fluid sample within the one or more chambers to be viewed from the exterior of the container. The apparatus may include a fill cap arranged to seal the internal space from the exterior of the container.

In accordance with a second aspect of the present invention, there is provided an apparatus for testing the quality of a fluid sample, the apparatus including:
a container defining an internal space for receiving the fluid sample;
a chamber disposed within the internal space for receiving a portion of the fluid sample;
a sealing element disposed within the internal space; and
an actuator for moving the sealing element from a spaced position, in which the sealing element is spaced from the chamber, to a sealing position, in which the sealing element seals the chamber,
wherein the apparatus includes an opening and the actuator includes a linkage arm that extends through the opening in a movably sealed manner, the linkage arm having a first portion coupled to the sealing element and a second portion disposed outside of the container, such that movement of the linkage arm from the exterior of the container causes movement of the sealing element.

Thus, the apparatus according to embodiments of the present invention enables one or more sealing elements disposed within the container to be actuated from outside of the container. This can improve the sealing force that can be applied to the sealing elements.

The apparatus may include an actuation member that is movably coupled to the outside of the body and coupled to the linkage arm such that movement of the actuation member causes movement of the linkage arm.

The actuation member may be rotatably coupled to the body and the apparatus may include one or more cam surfaces arranged to engage with the actuation member such that rotation of the actuation member causes the linkage arm to move axially.

The cam surfaces may be provided on one or more external faces of the container.

The opening may include an elongate shaft and the linkage arm may be coupled to a seal, the apparatus being arranged such that the seal remains within the shaft as the sealing element moves from the spaced position to the sealing position.

The apparatus may include a plurality of chambers and a plurality of sealing elements, each of which may be disposed within the internal space. The container may include one or more transparent or translucent portions for enabling the fluid sample within the one or more chambers to be viewed from the exterior of the container. The apparatus may include a fill cap for sealing the internal space from the exterior of the container.

In accordance with a third aspect of the present invention, there is provided an apparatus for testing the quality of a fluid sample, the apparatus including:
a chamber and having an opening for receiving at least some of the fluid sample; and
a sealing element arranged to seal the chamber,
wherein the apparatus includes a membrane arranged to permit oxygen to enter the chamber and arranged to inhibit the fluid sample from escaping from the chamber via the membrane, such that oxygen may enter the chamber while the chamber is sealed by the sealing element.

Thus, the apparatus according to embodiments of the present invention enables an aerobic environment to be maintained within one or more sealed chambers by providing one or more selectively-permeable membranes.

A wall defining the chamber may include the membrane.

The sealing element may include the membrane.

The apparatus may include a plurality of chambers. The apparatus may include a plurality of sealing elements. Each sealing element may be arranged to seal a respective chamber. The apparatus may include a plurality of membranes, each membrane being arranged to permit oxygen to enter a respective chamber and arranged to inhibit the fluid sample from escaping from the respective chamber via the membrane, such that oxygen may enter the respective chamber while the respective chamber is sealed by the sealing element.

The apparatus may include:
a container having an internal space for receiving the fluid sample; and
a fill cap for sealing the internal space from the exterior of the body,
wherein the chamber or chambers and sealing element or sealing elements are disposed within the internal space and wherein one of the container and cap includes a membrane arranged to permit oxygen to enter the internal space and inhibit the fluid sample from escaping from the internal space via the membrane, such that oxygen may enter the internal space while the container is sealed by the cap.

The membrane may include Gore-Tex™ or the like.

The membrane may include a region of the apparatus formed of silicone or the like.

The apparatus according to any of the aspects of the invention may be a water test apparatus, such as a field test kit. The apparatus according to any of the aspects of the invention may be arranged to enable the portion of the collected fluid sample isolated within each chamber to be tested for the presence of a substance while the apparatus is sealed from the outside environment.

### BRIEF DESCRIPTION OF THE DRAWINGS

Embodiments of the present invention will now be described, by way of non-limiting example only, with reference to the accompanying figures, in which:

FIG. 1 is a perspective view in cross section of apparatus according to a first embodiment of the present invention;

FIG. 2 is an exploded perspective view of the apparatus of FIG. 1;

FIG. 3 is a view in cross section of the apparatus of FIG. 1, showing the sealing elements in a spaced position permitting fluid communication between the secondary chambers and the primary chamber;

FIG. 4 is a view in cross section of the apparatus of FIG. 1, showing the sealing elements in a sealing position inhibiting fluid communication between the secondary chambers and the primary chamber;

FIG. 5 is a detailed view in cross section of a sealing element and secondary chamber of FIG. 1;

FIG. 6 is a view in cross section of apparatus according to a second embodiment of the present invention; and

FIG. 7 is a view in cross section of apparatus according to a third embodiment of the present invention.

### DETAILED DESCRIPTION

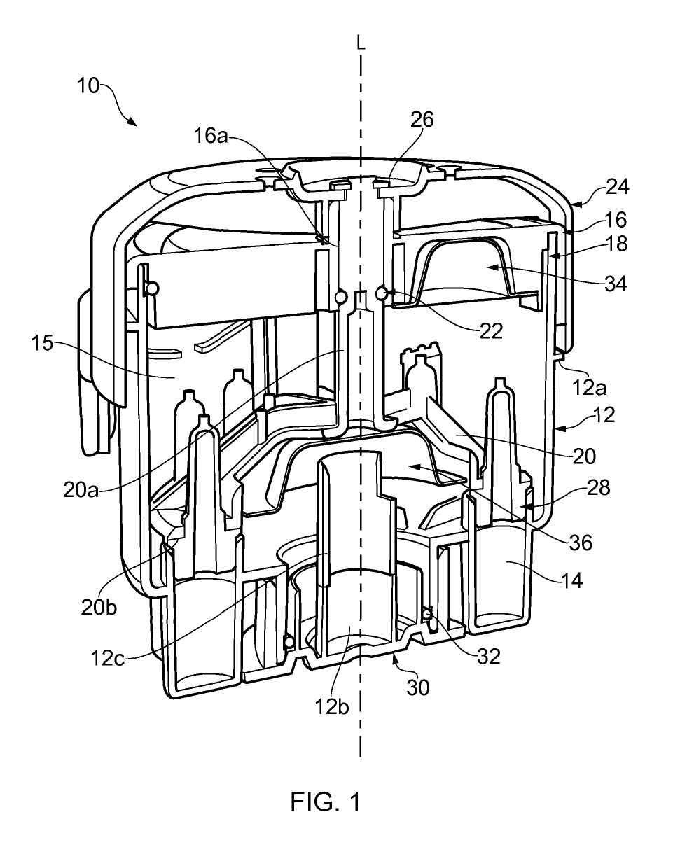

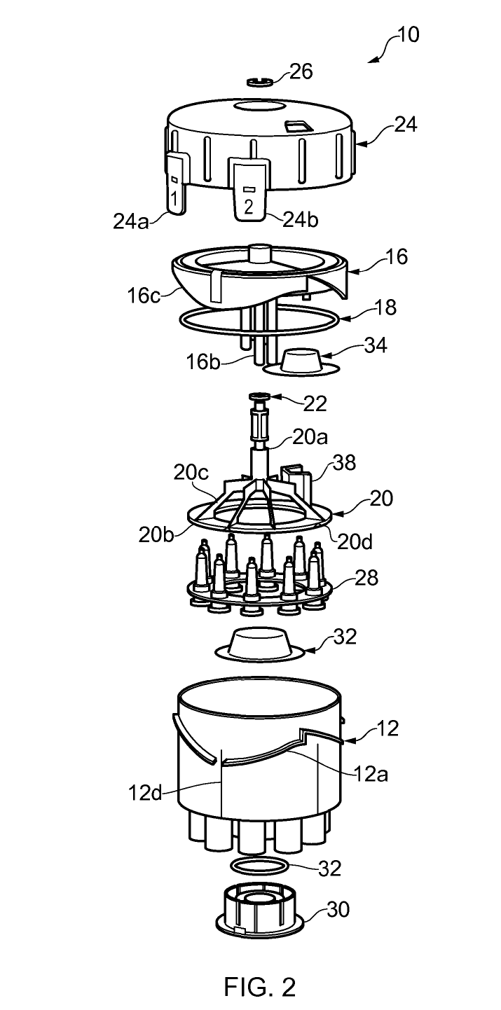

Referring to FIGS. 1 and 2, apparatus 10 for testing the quality of a fluid sample, such as a water sample, is shown according to a first embodiment of the invention. As an overview, the apparatus 10 includes a number of parts that together enable the apparatus 10 to be changed between a plurality of configurations. The apparatus include a body 12 which defines a plurality of chambers 14, 15. Each chamber 14, 15 has an opening. The chambers 14, 15 together define the internal space of the body 12. An inner lid 16 closes one end of the body 12. An outer lid 24 is arranged to move a sealing assembly 20, 28 disposed within the body 12. A fill cap 30 is provided seal the internal space of the body 12 from the outside environment.

The body 12 defines a primary chamber 15 and a plurality of secondary chambers 14. The volume of the primary chamber 15 is such it can hold a capacity of 90 ml of water. There are ten secondary chambers 14, arranged in a regular angular spacing around a longitudinal axis L of the body 12. Each secondary chamber 14 has a capacity of 1 ml of water. However, it is to be understood that the body 12 may, in other embodiments, have one or more chambers 14, 15 of any suitable capacity. The secondary chambers 14 are provided towards the base of the body 12. Each secondary chamber 14 has an opening through which it is in fluid communication with the primary chamber 15. Each secondary chamber 14 is generally cylindrical in shape. However, the secondary chambers may have any suitable configuration.

In the illustrated embodiment the body is unitary. However, in other embodiments the body 12 can be formed of two or more parts.

The secondary chambers 14 are arranged to be viewable from a fixed viewing angle in that at least some of the internal space of each secondary chamber 14 is viewable from the base. The base therefore forms a viewing portion. The primary chamber 15 is visible through one of the secondary chambers 14, as described in more detail below. However, in other embodiments the primary chamber 15 may include a centrally disposed primary chamber “well” that is viewable from the viewing portion, or in alternative embodiments the primary chamber 15 may be viewed through, for example, the said of the body 12, or through a transparent or translucent portion of the fill cap 30. In preferred embodiments the each secondary chamber 14 is viewable from the base.

The body 12 includes a primary opening 12b via which a fluid sample may be admitted to the internal space. The fill cap 30 houses an o-ring 32 and is arranged to couple with the body 12 to seal the primary opening 12b in a substantially fluid tight manner and as such the cap 30 is for sealing the internal space from the exterior of the body 12.

The seal assembly 20, 28 is movable between a first position where the secondary chambers 14 are each in fluid communication with the primary chamber 15, and a second position where one or more of the secondary chambers 14 are isolated from the primary chamber 15. The seal assembly 20, 28 includes a sealing structure 20 and a number of sealing elements 28. The sealing elements 28 are moulded as a single part and thus are joined together. However, in other embodiments the sealing elements 28 may be individual elements and/or integrally moulded with the seal plate 20b.

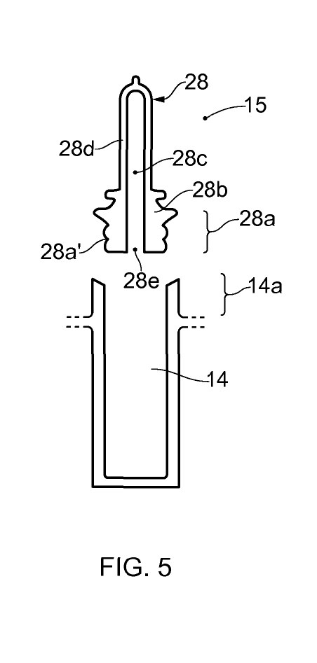

The configuration of the sealing elements 28 is described in more detail below, with reference to FIG. 5. In some embodiments the number of sealing elements 28 corresponds to the number of secondary chambers 14. In the illustrated example, each sealing element 28 except for one is arranged to isolate one of the secondary chambers 14 from the primary chamber 15, as well as from each other. The remaining sealing element (not shown) includes an opening arranged to enable fluid communication between its associated secondary chamber 14 and the primary chamber 15 when the seal assembly 20, 28 is in the second position, isolating the other secondary chambers 14, such that the fluid sample in the primary chamber 15 can be viewed through the associated, non-sealed, secondary chamber.

The sealing structure 20 includes an elongate rod 20a rigidly coupled to a seal plate 20b by a plurality of branches 20c. The rod 20a is slidably coupled to an outer lid 24 and arranged to moved therewith, as described in more detail below. The seal plate 20b extends in a circular path corresponding to the openings of the secondary chambers 14 and is coupled to the sealing elements 28. As such, the sealing elements 28 can be moved to engage with and seal the secondary chambers 14 through movement of the sealing structure 20. The sealing structure 20 is inhibited from rotating relative to the body 12 by a plurality of ribs 12d that project into the primary chamber 15 and extend generally parallel with respect to the longitudinal axis L, the ribs 12d being located within grooves 20d that extend radially into the circumferential face of the seal plate 20b. However, any suitable means may be provided to inhibit the seal assembly 20, 28 from rotating relative to the body 12.

The head of the body 12 is closed by an inner lid 16 that is coupled to the body 12 with an o-ring seal 18 there between, so as to form a substantially water-tight seal. The body 12 and inner lid 16 thus form a container, that can be formed as a single part. The inner lid 16 includes an opening which is preferably in the form of a shaft 16a that is generally coaxial with the longitudinal axis L of the body. The shaft 16a is arranged to slidably house the rod 20a of the sealing structure 20. The rod 20a includes a circumferential groove housing an o-ring seal 22. The apparatus 10 is arranged such that in normal used the o-ring seal 22 remains within the shaft 16a, such that a substantially water-tight seal is maintained as the seal assembly 20, 28 is moved between the first position and the second position.

The outer lid 24 is movably coupled to the body 12 and includes an opening within which a head portion of the rod 20a is rotatably coupled by a circlip 26, such that the rod 20a may rotate relative to the outer lid 24 about the longitudinal axis L but substantially may not move axially with respect outer lid 24. Thus, the rod 20a forms part of a linkage arm that extends through the shaft 16a in a movably sealed manner, the linkage arm having a first portion i.e. the seal plate 20b via which it is coupled to the sealing elements 28 and a second portion i.e. the head region of the rod 20a that is disposed outside of the body, such that movement of the linkage arm from the exterior of the body causes movement of the sealing elements 28. Thus, the outer lid 26 can be used to manipulate the seal assembly 20, 28.

The outer lid 24 includes a first catch 24a and a second catch 24b, each catching being arranged to inhibit the outer lid 24 rotating beyond a certain point unless a user lifts the respective catch 24a, 24b. However, in other embodiments the catches 24a, 24b may be omitted.

A cam surface 12a, 16c extends circumferentially around one or more portions of the body 12. In this embodiment the cam surface 12a, 16c comprises a lower cam track 12a disposed on the outside of the body 12 and an upper cam track 16c defined by a downwardly facing axial face of the inner lid 16. The outer lid 24 is arranged to engage with the cam surface 12a, 16c such that rotation of the cam surface 12a, 16c relative to the body 12 causes the outer lid 24 and thus the rod 20a to move axially. More specifically, rotation of the outer lid 24 through a sealing phase causes the upper cam track 16c to move the seal assembly 20, 28 downwards such that the sealing elements 28 seal the secondary chambers 14. Subsequent rotation of the outer lid 24 through an unsealing sealing phase causes the lower cam track 12a to move the seal assembly 20, 28 away from the chambers 14, towards the inner lid 16. However, it will be appreciated that in other embodiments the seal assembly 20, 28 may be actuated in any suitable manner. For example, the rod 20a may include a threaded portion rotatably coupled to a threaded portion on the shaft 16a. In other embodiments the outer lid 24 may be dispensed with all together and the head portion of the rod 20a can define a handle arranged for direct manipulation by a user. Providing the cam surface 12a, 16c, or other suitable actuation means, outside the apparatus 10 is advantageous because it allows embodiments of the invention to increase the amount of axial travel performed by the seal assembly 20, 28 that would be the case if the actuation means is disposed within the primary chamber 15. Also, the parts defining the actuation surfaces may in some cases be larger because they do not have to fit within the primary chamber 15.

A reagent retaining means 32 is provided between the sealing structure 20 and the fill cap 30 and retained in position by the downwardly protruding fingers 16b of the inner lid 16. The reagent retaining means 32 may be a blister pack or other moisture-resistant packaging. Reagent and growth media may be provided in the reagent retaining means 32. In some embodiments a fluorogenic reagent may be used, such as 4-Methylumbelliferyl-β-D-glucuronide (MUG). However, it should be noted that any reagent may be used that is suitable for testing the quality of a fluid sample, for example a chromogenic reagent such as 5-bromo-4-chloro-3-indolyl-β-D-glucuronide (X-gluc). In some embodiments one or more functional agents or additives may be provided along with the reagent, for example a growth media including carbon sources such as yeast extract or peptone, one or more salts or buffers to maintain a pH close to neutral, an additive such as sodium dodecyl sulphate to suppress the growth of gram-positive bacteria, or an additive such as sodium thiosulfate to neutralise any chlorine residual.

The body 12 includes a movable reagent piercing member 12c arranged to pierce the reagent retaining means 32 when the fill cap 30 is coupled to the body 12, due to the fill cap forcing the movable reagent piercing member 12c into the reagent retaining means 32. It is however to be understood that, in embodiments where the apparatus includes reagent and/or growth media, any suitable means may be used to release any suitable reagent and/or growth media.

A decontaminant retaining means 34 is provided on the underside of the inner lid 16. The decontaminant retaining means 34 may be a blister pack or other moisture-resistant packaging. A substance such as Virkon® or sodium hypochlorite may be retained in the decontaminant retaining means 34 by, for example, foil or other moisture-resistant packaging.

A decontaminant piercing member 38 extends upwardly from the sealing structure 20 and moves therewith such that it may pierce the decontaminant retaining means 34. It is however to be understood that, in embodiments where the apparatus includes a decontaminant, any suitable means may be used to release any suitable decontaminant.

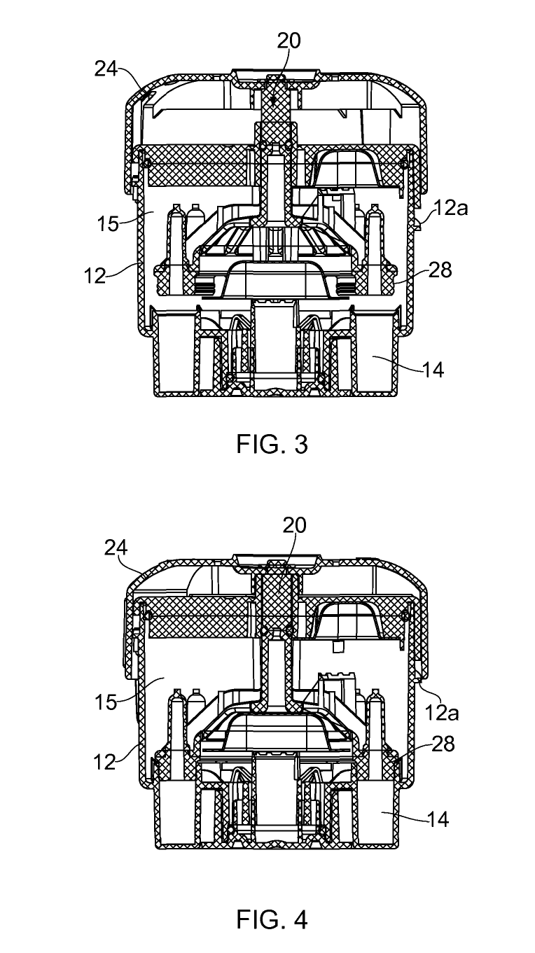

In use, with the sealing assembly 20, 28 in the first position, as shown in FIG. 3, a water sample is introduced into the internal space of the body 12. The fill cap 30 is then coupled to the body 12, to seal the internal space, which in turn punctures the reagent retaining means 32. A user may shake the apparatus 10 to mix the reagent and/or growth media in the water sample. The first catch 24a is lifted to enable the outer lid 24 to be rotated relative to the body 12. Doing so causes the outer lid 24 to be forced towards the body 12 by the upper cam track 16c, which in turn axially forces the seal assembly 20, 28 towards the secondary chambers 14 such that the sealing elements 28 seal portions of the collected water sample within the secondary chambers 14, with the remaining portion of the water sample being sealed in the primary chamber 15, as shown in FIG. 4. Thus, the sealing elements 28 are arranged to be movable to a sealing position in which an insertion portion 28a of a respective sealing element 28 is received within an insertion subspace 14a of the chamber space of a respective secondary chamber 14 to seal the respective chamber opening. The apparatus 10 may then be incubated for a period of time, after which the isolated samples within the chambers 14, 15 can be read to determine the quality of the collected water sample. Following this, the second catch 25b may be lifted, thereby enabling the user to further rotate the outer lid 24. Such rotation causes the cam surface 12a, 16c to lift the outer lid 24 and thus the seal assembly 20, 28 away from the secondary chambers 14, so as to cause the decontaminant piercing member 38 to pierce the decontaminant retaining means 34.

Referring additionally to FIG. 5, a sealing element 28 of the apparatus 10 is shown. However, the following description relating to FIG. 5 is equally applicable to other sealing elements 28 of the apparatus 10 that are arranged to seal a secondary chamber 14. The sealing element 28 includes a body 28b having a peripheral sealing face 28a′ which forms the insertion portion 28a of the sealing element 28. The body 28b is formed of a resiliently deformable material, such as a thermoplastic elastomer. The peripheral sealing face 28a′ includes circumferentially extending ribs that may aid the seal formed between the sealing element 28 and wall defining the chamber 14.

The sealing element 28 includes a displacement passage in the form of an elongate auxiliary chamber 28c having an opening to enable fluid communication with the insertion space 14a and is arranged to enable a portion of the fluid sample located within the insertion space 14a to be displaced into the auxiliary chamber 28c as the sealing element 28 assumes the sealing position. Preferably the auxiliary chamber opening 28e, which is provided at the face of the sealing element 28, is sized such that the fluid sample will enter the opening 28e as the sealing element 28 assumes the sealing position, but substantially will not enter the opening 28e before this, i.e. when the sealing element 28 is spaced from the chamber 14, due to surface tension of the fluid. This is advantageous because, in some cases, the sealing element 28 will be moving to the sealing position while the chambers 14, 15 contain the fluid sample. The chamber opening 28e traps gas, such as air, in the auxiliary chamber 28c and this gas can be compressed as the fluid sample is forced into the auxiliary chamber 28c from the insertion space 14a as the sealing element 28 assumes the sealing position.

The sealing element 28 includes a head portion 28d defining a portion of the auxiliary chamber 28c. The head portion 28d is arranged so as not to enter the chamber 14 when the sealing element 28 is in the sealing position. This is advantageous because it means that a large portion of the secondary chamber 14 can be used for holding some of the fluid sample that would be the case if the head portion 28d entered the chamber 14. At least some of the head portion 28d is formed of a resiliently deformable material, such as a thermoplastic elastomer, such that the volume of the auxiliary chamber 28c may increase with an increase of fluid pressure within the sealed chamber space 14. The thickness of the sealing element 28 at the head region 28d is less than the thickness of the sealing element 28 at the insertion portion 28a to facilitate this. The expansion capability may in some embodiments be improved because the head region 28d expands into the primary chamber 15 rather than into the secondary chamber 14. The relatively thicker insertion portion 28a may aid in moving the sealing element 28 to the sealing position.

The sealing element 28 is arranged such that an increase of fluid pressure within the sealed chamber space 14, which includes auxiliary chamber 28c, results in net pressure acting against the chamber walls that define the insertion subspace 14a of the chamber 14. This is advantageous because, in some cases, a subsample that is isolated within a sealed chamber 14 will experience a pressure increase due to growth of substances within the subsample. Due to this, a known, flat faced, solid sealing element may be forced away from the secondary chamber 14, thereby enabling some of the isolated sub sample to escape from the secondary chamber 14. However, in embodiments of the present invention, such a pressure increase results in expansion of the head region 28d, which results in the peripheral sealing face 28a′ being forced against the chamber walls that define the insertion subspace 14a of the chamber space.

In some embodiments the apparatus 10 may have any suitable configuration wherein it includes a chamber having an opening for receiving at least some of the fluid sample and

a sealing element arranged to be movable to a sealing position in which an insertion portion of the sealing element is received within an insertion space of the chamber to seal the opening, wherein one of the sealing element and container is arranged to define a displacement passage for enabling a portion of the fluid sample corresponding in volume to that which is located within the insertion space to be displaced therefrom as the sealing element assumes the sealing position. Such an embodiment may closely resemble the apparatus shown in FIG. 5. In some embodiments the displacement passage may be defined within the walls of the chamber and include means arranged to inhibit entry of the fluid sample during normal mixing conditions, but permit the entry of a quantity of fluid corresponding to that present in the insertion space as the sealing element assumes the sealing position.

In some embodiments, the displacement passage may be defined by a resiliently deformable displacement region of the sealing element or chamber, the resiliently deformable displacement region being arranged to deform as the sealing element assumes the sealing position to define a passageway for enabling the displacement of the portion of the fluid sample within the insertion space. For example, the chamber walls that defines the insertion subspace 14a of the chamber space may be formed of a resiliently deformable material, such as a thermoplastic elastomer or silicone, so as to form the resiliently deformable displacement region.

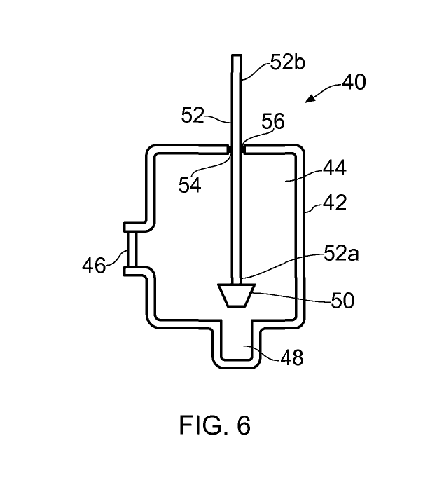

Referring to FIG. 6, in a second embodiment the apparatus 40 may have any suitable configuration wherein it includes a body 42 having an internal space 44 for receiving the fluid sample, a cap 46 for sealing the internal space 44 from the exterior of the body 42, a chamber 48 disposed within the internal space 44 for receiving a portion of the fluid sample, a sealing element 50 disposed within the internal space 44 and an actuator 52 for moving the sealing element 50 from a spaced position, in which the sealing element 50 is spaced from the chamber 48, to a sealing position, in which the sealing element 50 seals the chamber 48, wherein the apparatus 40, for example the body 42, includes an opening 54 and the actuator 52 includes a linkage arm 52 that extends through the opening 54 in a movably sealed manner, for example due to o-ring 56, the linkage arm 52 having a first portion 52a coupled to the sealing element 50 and a second portion 52b disposed outside of the body 42, such that movement of the linkage arm 52 from the exterior of the body 42 causes movement of the sealing element 50. In some embodiments the cap 46 may be omitted. As will be appreciated by a skilled person, any suitable feature or combination of features from the embodiments described with reference to FIGS. 1 to 5 may be included in the apparatus 40 of the second embodiment.

In the embodiment illustrated with reference to FIGS. 1 to 5, at least some of the walls that define the secondary chambers 14 are formed of silicone, or another material of similar oxygen permeability such as a PTFE membrane. The silicone defines a membrane arranged to permit oxygen to enter the chamber 14 yet inhibit the fluid sample from escaping from the chamber 14 via the membrane, such that oxygen may enter the chamber 14 while the chamber 14 is sealed by the sealing element 28. More specifically, in some embodiments the membrane is arranged to prevent microbiological cross contamination through it, yet permit oxygen transfer. Providing such a selectively-permeable membrane according to embodiments of the invention is advantageous because it creates an aerobic environment within the sealed chambers 14 that may support the use of chromogenic reagents that require oxygen to produce a chromogenic effect. Also, the aerobic environment within the sealed chambers 14 may be advantageous when using fluorogenic reagents such as Resorufin because the oxygen may enhance such reagents. Also, the aerobic environment within the sealed chambers 14 may be advantageous because it enables the growth of organisms that require an aerobic environment.

In some embodiments, a portion of the apparatus may include a breathable barrier material, such as Gore-Tex™.

In some embodiments, one of the body 12 and cap 30 may include a membrane arranged to permit oxygen to enter the primary chamber 15 and inhibit the fluid sample from escaping from the internal space via the membrane, such that oxygen may enter the primary chamber 15 while the chamber is sealed by the cap 30. In such a case, one or more of the sealing elements 24 may include a membrane to permit oxygen within the primary chamber 15 to pass into the secondary chambers 14, while isolating the fluid subsamples within the chambers 14.

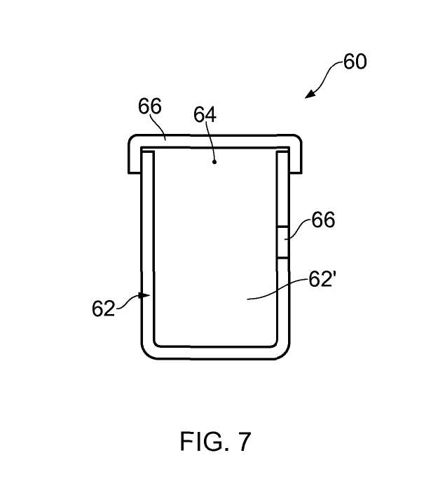

Referring to FIG. 7, in a third embodiment the apparatus 60 may have any suitable configuration wherein it includes a chamber 62 having an opening 64 for receiving at least some of the fluid sample and a sealing element 66 arranged to seal the chamber 62, wherein the apparatus includes a membrane 66 arranged to permit oxygen to enter the chamber 62 and inhibit the fluid sample from escaping from the chamber 62′ via the membrane 66, such that oxygen may enter the chamber 62 while the chamber 62 is sealed by the sealing element 66. As will be appreciated by a skilled person, any suitable feature or combination of features from the embodiments described with reference to FIGS. 1 to 5 may be included in the apparatus 60 of the third embodiment.

1. Apparatus for testing the quality of a water sample, the apparatus including:
a chamber having a chamber wall having an opening for receiving at least some of the water sample; and
a sealing element arranged to be movable to a sealing position in which an insertion portion of the sealing element is received within an insertion space of the chamber to seal the opening,
wherein one of the sealing element and chamber wall is arranged to define a displacement passage for enabling a portion of the water sample that is located within the insertion space to be displaced therefrom as the sealing element assumes the sealing position.

2. Apparatus according to claim 1, wherein the sealing element includes a body portion having a peripheral sealing face which forms at least some of the insertion portion of the sealing element.

3. Apparatus according to claim 1, wherein at least the insertion portion of the sealing element is formed of a resiliently deformable material.

4. Apparatus according to claim 1, wherein the displacement passage is formed in the sealing element and includes a second opening leading to an auxiliary chamber arranged to enable the portion of the fluid sample located within the insertion space to be displaced into the auxiliary chamber as the sealing element assumes the sealing position.

5. Apparatus according to claim 4, wherein the second opening is arranged to permit water of the water sample to enter the auxiliary chamber when at a first pressure corresponding to the pressure within the chamber as the sealing element assumes the sealing position, and arranged to inhibit water of the water sample from entering the auxiliary chamber when at a second pressure less than the first pressure and corresponding to the fluid pressure within the chamber when the sealing element is spaced from the container.

6. Apparatus according to claim 4, wherein the sealing element includes a head region that defines a portion of the auxiliary chamber, the head region being arranged so as not to enter the chamber when the sealing element is in the sealing position.

7. Apparatus according to claim 6, wherein at least some of the head region is formed of a resiliently deformable material such that the volume of the auxiliary chamber may increase with an increase of fluid pressure within the chamber while the chamber is sealed by the sealing element.

8. Apparatus according to claim 1, wherein the sealing element is arranged such that net pressure resulting from an increase in fluid pressure within the sealed chamber acts against the container walls that define the insertion subspace of the chamber.

9. Apparatus according to claim 1, wherein the displacement passage is defined by a resiliently deformable displacement region of the sealing element or chamber wall, the resiliently deformable displacement region being arranged to deform as the sealing element assumes the sealing position to define a passageway for enabling the displacement of the portion of the fluid sample within the insertion space.

10. Apparatus according to claim 9, wherein at least some of the chamber wall is formed of a resiliently deformable material to form the resiliently deformable displacement region.

11. Apparatus according to claim 1, wherein the apparatus includes a container defining an internal space for receiving the fluid sample, the chamber and sealing element being disposed within the internal space such that the chamber is filled with the fluid sample as the sealing element moves to the sealing position.

12. Apparatus for testing the quality of a fluid sample, the apparatus including:
a container defining an internal space for receiving the fluid sample;
a chamber disposed within the internal space for receiving a portion of the fluid sample;
a sealing element disposed within the internal space; and
an actuator for moving the sealing element from a spaced position, in which the sealing element is spaced from the chamber, to a sealing position, in which the sealing element seals the chamber,
wherein the apparatus includes an opening and the actuator includes a linkage arm that extends through the opening in a movably sealed manner, the linkage arm having a first portion coupled to the sealing element and a second portion disposed outside of the container, such that movement of the linkage arm from the exterior of the container causes movement of the sealing element.

13. Apparatus according to claim 12, including an actuation member movably coupled to the outside of the body and coupled to the linkage arm such that movement of the actuation member causes movement of the linkage arm.

14. Apparatus according to claim 13, wherein the actuation member is rotatably coupled to the body and the apparatus includes one or more cam surfaces arranged to engage with the actuation member such that rotation of the actuation member causes the linkage arm to move axially.

15. Apparatus according to claim 14, wherein the cam surfaces are provided on one or more external faces of the container.

16. Apparatus according to claim 12, wherein the opening includes an elongate shaft and the linkage arm is coupled to a seal, the apparatus being arranged such that the seal remains within the shaft as the sealing element moves from the spaced position to the sealing position.

17. Apparatus according to claim 12, including a cap for sealing the internal space from the exterior of the apparatus.

18. Apparatus for testing the quality of a fluid sample, the apparatus including:
a chamber and having an opening for receiving at least some of the fluid sample; and
a sealing element arranged to seal the chamber,
wherein the apparatus includes a membrane arranged to permit oxygen to enter the chamber and arranged to inhibit the fluid sample from escaping from the chamber via the membrane, such that oxygen may enter the chamber while the chamber is sealed by the sealing element.

19. Apparatus according to claim 18, wherein a wall defining the chamber includes the membrane.

20. Apparatus according to claim 18, wherein the sealing element includes the membrane.

21-25. (canceled)
